(19) United States
(12) Patent Application Publication
(10) Pub. No.: US 2013/0101986 A1
(43) Pub. Date: Apr. 25, 2013

(54) In Vitro Method for obtaining Intrahepatic Fibroblasts Infected with Hepatitis C Virus

(71) Applicants: Yvon Calmus, Paris (FR); Filomena Conti, Velizy (FR)

(72) Inventors: Yvon Calmus, Paris (FR); Filomena Conti, Velizy (FR)

(21) Appl. No.: 13/501,856

(22) PCT Filed: Oct. 15, 2010
(86) PCT No.: PCT/IB10/54687

§ 371 Date: Nov. 30, 2012

(30) Foreign Application Priority Data
Oct. 16, 2009 (FR) 0904962

(51) Int. Cl. C12Q 1/02

(52) U.S. Cl. CPC C12Q 1/025
USPC 435/5; 435/325; 435/235.1

(57) ABSTRACT

The present invention relates to in vitro methods for obtaining intrahepatic fibroblasts infected with hepatitis C virus, to the infected intrahepatic fibroblasts obtained by means of these methods, and also to methods for screening for anti-fibrogenesis molecules and for anti-HCV molecules using these cells.

The hepatitis C virus is an enveloped virus with a single-stranded RNA genome of positive polarity. It belongs to the genus Hepacivirus, of which it is the only member, and forms part of the family Flaviviridae. The replication of the genomic RNA passes through the intermediate stage of an antigenomic strand of negative polarity, which will in its turn serve as template for the synthesis of genomic RNAs. The neosynthesized genomic RNAs will serve either for the synthesis of a polyprotein cleaved co- and post-translationally by cellular and viral proteases to produce the structural and nonstructural viral proteins, or for the synthesis of new strands of RNA of negative polarity, or else will be encapsidated following oligomerization of the capsid protein. The nucleocapsids formed in the cytoplasm acquire their envelope by budding at the level of the endoplasmic reticulum, where the glycoproteins E1 and E2 are retained, and then the viral particles are released into the external environment.

Infection with this virus is an important public health problem in view of the high prevalence of infection and of the considerable risk of progression to chronic hepatitis, estimated at between 50 and 80%. In fact, the hepatitis C virus (HCV) infects about 3% of the population and is responsible for approximately 170 million chronic infections. The chronic infection gradually leads to fibrous hepatitis, which may progress to cirrhosis and to liver cancer.

At present, the most effective antiviral treatment consists of bitherapy based on the combined use of pegylated interferon alfa and a nucleoside analog, ribavirin. However, this treatment is only effective in about 50% of patients treated, and moreover is poorly tolerated by many patients.

Combating the hepatitis C virus is therefore still a major public health challenge, and it is essential to develop novel molecules targeting the viral cycle, or capable of stopping, or even preventing, the occurrence of fibroses induced during HCV infection.

Hepatic fibrosis is a common complication of chronic liver diseases, and especially of viral hepatitis C. It is characterized by accumulation of extracellular matrix (ECM) composed principally of collagens of type I, III, IV and V. In the course of hepatic fibrogenesis, hepatic stellate cells (HSCs) and intrahepatic fibroblasts (IHFs) are activated or are transdifferentiated into hepatic myofibroblasts, which acquire the ability to express smooth muscle alpha-actin, which produce extracellular matrix (ECM) and inhibit the degradation of the latter by degrading the metalloproteases.

The mechanism of the hepatic lesions induced during infection with the hepatitis C virus (HCV) is poorly understood and the link between infection of hepatocytes with this virus and activation of fibrogenesis is still hypothetical. It has notably been proposed that chronic inflammation resulting from infection of hepatocytes with HCV produces a stimulus that induces activation of the intrahepatic fibroblasts (IHFs). According to this hypothesis, cytokines released by HCV-infected hepatocytes, endothelial cells, Kupffer cells or infiltrated lymphocytes might be responsible for the activation (Friedman S L., J. Biol. Chem., 275: 2247-2250, 2000; Schulze-Krebs et al., Gastroenterology, 129(1): 246-258, 2005).

In order to develop antivirals effectively targeting the fibrosis induced during HCV infection, it is necessary to elucidate the mechanisms at the origin of this virus-induced fibrosis.

The inventors have now discovered that HCV is capable of infecting intrahepatic fibroblasts (IHFs) in vivo and activating them, which strongly suggests that the hepatocytes might act as a reservoir of the virus, but that the fibrosis would be due essentially to infection of the fibroblasts. This discovery is surprising since up to now HCV has only been known to infect hepatocytes, monocytes, lymphocytes and certain secretory cells (Shimizu et al., Hepatology, 23: 205-209, 1996; Wong et al., J. Virol., 75: 1229-1235, 2001; Caussin-Schwemling et al., J. med. Virol., 65: 14-22, 2001; Arrieta et al., Am. J. Pathol., 158: 259-264, 2001). There are no data that would suggest that the intrahepatic fibroblasts are sensitive and accessible to HCV infection.

Thus, a first object of the present invention relates to a method of obtaining intrahepatic fibroblasts infected with the hepatitis C virus comprising a step of isolating the intrahepatic fibroblasts from hepatic tissue obtained from an HCV-positive patient.

“HCV-positive patient” means any person who, on the day of taking the sample of hepatic tissue, is infected with HCV. It can notably be a patient who has chronic hepatitis C.

The intrahepatic fibroblasts can be isolated by various methods that are known per se, for example that described by Tiggelman M B C et al. (J. Hepatol., 23: 307-317, 1995) or that described by Win K M et al. (Hepatology, 18: 137-145, 1993). These cells can easily be characterized, and differentiated from other cellular types, notably by the method described by Aoudjehane et al. (Lab. Invest., 88: 973-985, 2008). Moreover, as the intrahepatic fibroblasts are activated cells expressing smooth muscle alpha-actin and vimentin, these markers can be used for characterizing them, in association with the presence of the surface molecule CD90, and absence of the molecule CD31.

The inventors also found that it was possible to infect intrahepatic fibroblasts with HCV in vitro. The invention therefore also relates to another method of obtaining intrahepatic fibroblasts infected with the hepatitis C virus comprising a step of contacting intrahepatic fibroblasts in vitro with infectious particles of the hepatitis C virus. The stocks of infectious viral particles originate from patients' sera or from culture of the virus in vitro. In the latter case, the infectious particles can notably be the HCV-JFH1 particles produced by transfecting the genomic RNA transcribed from the plasmid pJFH-1 in Huh-7.5 cells (Wakita et al., Nat. Med., 11: 791-796, 2005). Infectious particles other than HCV-JFH1, expressing different genotypes or chimeric genotypes, can also be used, notably those described by Gottwein et al. (Gastroenterology, 133: 1614-1626, 2007) and Pietschmann et al. (P.N.A.S., 103: 7408-7413, 2006).

The intrahepatic fibroblasts infected with the hepatitis C virus, notably those obtained by the methods described above, also form part of the present invention. “Intrahepatic fibroblasts infected with the hepatitis C virus” means not only the intrahepatic fibroblasts infected following attachment of the virus to the cell and penetration of the viral particle into the cell, but also any cell comprising the genomic RNA (strand of positive polarity) and/or antisense RNA (strand of negative polarity) of HCV, or any other RNA comprising all or part of the genomic RNA of HCV and conserving the capacity to replicate in the cell, for example bicistronic replicons described by Lohmann et al. (Science, 285: 110-113, 1999).

Another object of the present invention relates to a method of in vitro replication of the genome of the hepatitis C virus that comprises a step of in vitro culture of isolated intrahepatic fibroblasts infected with the hepatitis C virus. Advantageously, the isolated infected intrahepatic fibroblasts are obtained using the methods according to the invention as defined above. The optimal culture conditions can easily be determined by a person skilled in the art by routine operations.

Moreover, as the intrahepatic fibroblasts are infected in vivo and are responsible for hepatic fibrosis, it is essential to test the infected intrahepatic fibroblasts as targets of anti-HCV drugs or of drugs aiming to reduce the hepatic lesions induced by HCV, and in particular fibrosis.

The invention therefore also relates to a method of screening anti-HCV molecules comprising the following steps:
putting intrahepatic fibroblasts infected with a hepatitis C virus, as defined above, in contact with the molecule to be tested;
measuring the replication of the HCV genome and/or the production of HCV particles, notably in the culture supernatant.

When measuring the replication of the viral genome, the RNA strand of positive polarity and/or the RNA strand of negative polarity of HCV are quantified.

The production of viral particles can be measured by various methods that are known per se, for example techniques of immunodetection, notably by quantification of the structural proteins (C, E1, E2) in the supernatant, or by observation in electron microscopy after density gradient enrichment (Lindenbach B D et al., Science, 309: 623-626, 2005).

The invention also relates to a method of screening antifibrogenesis molecules comprising the following steps:
putting intrahepatic fibroblasts infected with a hepatitis C virus as defined above in contact with the molecule to be tested;
measuring the expression, by said intrahepatic fibroblasts, of at least one gene involved in fibrosis.

“Gene involved in fibrosis” means any gene that is expressed in the activated intrahepatic fibroblasts and that contributes to the fibrogenesis responsible for hepatic fibrosis (for a review see the publication World. J. Gastroenterol., 15(20): 2433-2440, May 28, 2009, and notably Table 1). We may notably mention the genes of collagens of type I, III, IV and V.

Advantageously, the above two methods of screening further comprise a step of comparing the measurement result obtained with HCV-infected intrahepatic fibroblasts brought into contact with the molecule to be tested with that obtained with HCV-infected intrahepatic fibroblasts that have not been put in contact with the molecule to be tested.

The main classes of drugs suitable for testing are notably i) antivirals, capable of reducing the infection of the hepatocytes, but whose effect on fibroblasts is unknown, ii) antifibrosing drugs that have only been tested on uninfected fibroblasts, iii) immunosuppressants, capable of modulating both the replication of HCV and the activation of fibroblasts.

Moreover, as the intrahepatic fibroblasts are infected in vivo and are responsible for hepatic fibrosis, it is at least as important to consider the IHFs as the hepatocytes for testing the toxicity of drugs that will be used in patients who are carriers of HCV. The invention therefore further relates to a method of evaluating the toxicity of a molecule with respect to hepatic fibroblasts of a patient with hepatitis C comprising the following steps:
putting intrahepatic fibroblasts infected with a hepatitis C virus or intrahepatic fibroblasts comprising the genome of a hepatitis C virus or a replicon thereof in contact with the molecule to be tested;
evaluating the mortality of said intrahepatic fibroblasts put in contact with the molecule to be tested.

Cellular mortality means apoptosis and/or necrosis of cells. The techniques for evaluating cellular mortality are well known per se. These are notably flow cytometry after labeling with annexin V and propidium iodide, and the TUNEL technique (immunocytochemistry), techniques that are described in the article by Aoudjehane et al. (FASEB J., 21(7): 1433-1444, 2007).

Advantageously, the intrahepatic fibroblasts on which the toxicity of a molecule is tested are intrahepatic fibroblasts infected with the hepatitis C virus as defined above.

The present invention will be better understood from the rest of the description given hereunder, which refers to nonlimiting examples notably illustrating the isolation of intrahepatic fibroblasts (IHFs) infected in vivo with the hepatitis C virus, the infection in vitro of IHFs with HCV, and demonstration that infection of intrahepatic fibroblasts with HCV induces expression of collagens I, III and IV.

### EXAMPLE 1

### Isolation and Culture of Human Intrahepatic fibroblasts (IHFs)

Intrahepatic fibroblasts (IHFs) were isolated from hepatic tissues obtained either from patients who had undergone hepatectomy for benign metastases or tumors, or from patients with chronic infection with the hepatitis C virus and who have developed liver cancer or some other type of tumors. The hepatic tissues taken during major hepatectomy were as remote as possible from the tumor, avoiding the immediate peritumoral zone. Dissociation of the tissues was carried out by a method of two-stage collagenase perfusion, the cells being separated by gradient centrifugation, as described by Hillaire et al. (Gastroenterology, 107: 781-788, 1994).

The visible vessels were first perfused with HEPES-EDTA buffer, then with Liver Digest solution (Gibco, Cergy-Pontoise, France) comprising 0.05% of collagenase, at a rate of 10 ml per catheter per minute for 30 minutes. The fragments of liver were then agitated gently in order to release the detached hepatic cells, then filtered and centrifuged. The hepatocytes were isolated from the pellet, whereas the intrahepatic fibroblasts were isolated from the supernatant. The intrahepatic fibroblasts were separated from the other cells of the supernatant by centrifugation at 1800 rpm for 10 minutes as described by Win K m et al. (Hepatology, 18: 137-145, 1993). The cellular viability was determined by the trypan blue exclusion test, and the intrahepatic fibroblasts were seeded in a culture dish in DMEM medium (Gibco) supplemented with 10% of fetal calf serum, 100 U/ml of penicillin and 100 mg/ml of streptomycin and cultivated at 37° C. under an atmosphere of 5% CO₂. The culture medium was replaced one day after seeding. At confluence, the cells are cultivated again and kept in a 75 cm² flask.

The purity of the intrahepatic fibroblasts in culture was analyzed by flow cytometry, immunohistochemistry and immunofluorescence as described previously by Aoudjehane et al. (Lab. Invest., 88: 973-985, 2008). Thus, the purity and the activation of the intrahepatic fibroblasts were evaluated by measuring the expression of smooth muscle alpha-actin, a marker of hepatic stellate cells, and of vimentin, a marker of cells of mesenchymal origin. In addition, the intrahepatic fibroblasts were also characterized by flow cytometry and immunofluorescence by measuring the expression of CD90, a marker of fibroblasts, and of CD31, a marker of endothelial cells. More than 95% of the cells possessed the marker CD90 and only 1% of the cells had the marker CD31, which indicates that the culture of intrahepatic fibroblasts isolated by the method described above is almost pure.

In the experiments described below, only the cells obtained from passages two to six were used. Moreover, each experiment was conducted using cells obtained from at least six different human livers.

### EXAMPLE 2

### Evaluation of the Expression of CD81 and of the LDL Receptor (LDL-R) on the Surface of Human Intrahepatic Fibroblasts (IHFs)

The expression of CD81 and of the receptor LDL-R was evaluated by immunohistochemistry and flow cytometry.

2.1. Evaluation by Immunohistochemistry

The intrahepatic fibroblasts were grown on slides in 6-well plates (2.10⁴ cells per well) in DMEM medium (Gibco) supplemented with 10% of fetal calf serum and then fixed in PBS containing 4% of paraformaldehyde. Alternatively, when the cells are grown on plates, the cells are resuspended in PBS and then cytocentrifuged on SuperFrost Plus Slides (CML, Nemours, France). The cells are then dried in air overnight at room temperature, fixed for 10 minutes with acetone, then used directly or stored at −20° C. until analysis by immunohistochemistry.

The expression of the CD81 surface molecule and of the LDL-R receptor was investigated by an indirect immunoenzymatic technique using an alkaline phosphatase/anti-alkaline phosphatase complex as described previously by Conti et al. (Transplantation, 76: 210-216, 2003). Briefly, CD81 was detected by means of an anti-CD81 monoclonal antibody diluted 1/50 used as primary antibody and a rabbit anti-mouse IgG used as secondary antibody. For detecting the receptor LDL-R, anti-LDL-R polyclonal antibodies diluted to 1/10 and mouse anti-rabbit IgGs were used respectively as primary and secondary antibody. The slide was then incubated in the presence of alkaline phosphatase/anti-alkaline phosphatase complexes, and the alkaline phosphatase activity was developed for 20 minutes in solutions of fast-red TR (1 mg/ml) and of naphthol phosphate (0.2 mg/ml) (Sigma, Saint Quentin-Fallavier, France) containing 0.24 g/ml of levamisole. To finish, the slides were counterstained with hematoxylin (nuclear stain). Negative controls, for which the primary antibody was omitted or was replaced with a nonspecific antibody, were also analyzed. Moreover, a positive control, namely PBMC cells that naturally express the CD81 molecule and LDL-R on their surface, was also analyzed.

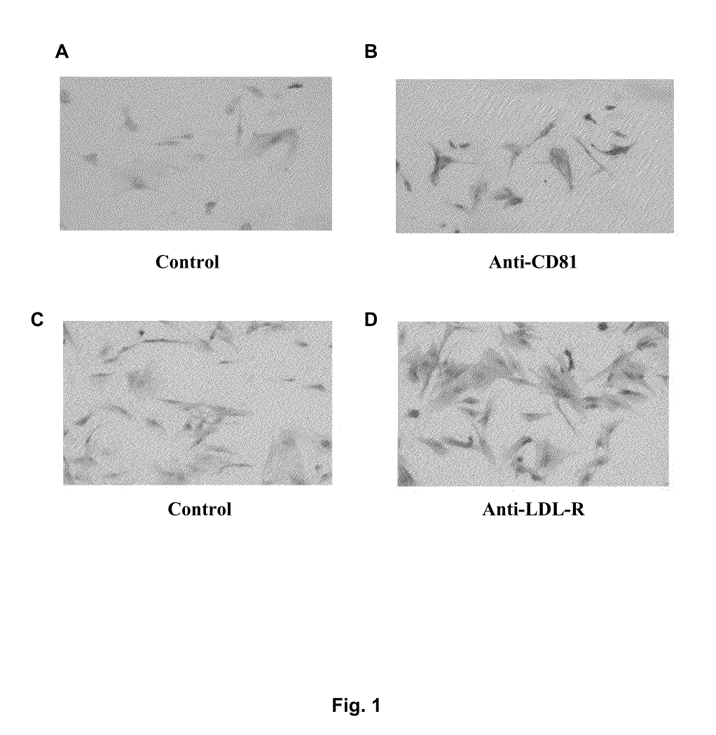

The results of the study by immunohistochemistry are presented in FIG. 1. FIGS. 1.A to 1.C are black-and-white photographs of intrahepatic fibroblasts fixed on control slides (not treated with the anti-CD81 or anti-LDL-R antibodies), and FIGS. 1.B and 1.D show the results obtained for intrahepatic fibroblasts fixed on slides treated with anti-CD81 and anti-LDL-R antibodies respectively.

This diagram shows that the control cells are not colored (they appear light gray in FIGS. 1.A and 1.C) whereas the cells put in contact with an anti-CD81 antibody or an anti-LDL-R antibody are colored (they appear dark gray in FIGS. 1.B and 1.D), which indicates that the antibodies have become fixed on the cell surface.

These results therefore show that the intrahepatic fibroblasts express the CD81 molecule and LDL-R, the two putative receptors of HCV, on the cell surface.

It should be noted that, as expected, the PBMC cells (positive control) were labeled with the anti-CD81 and anti-LDL-R antibodies (results not shown).

2.2. Evaluation by Flow Cytometry

The expression levels of the CD81 molecule and of LDL-R were also analyzed by flow cytometry. For this purpose, the cells were contacted with a rabbit anti-human LDL-R antibody or with a mouse anti-human CD81 antibody for 60 minutes at 4° C. The cells were then washed and incubated with an antirabbit or anti-mouse secondary antibody respectively. The cells were then washed in PBS and then fixed in PBS containing 4% of paraformaldehyde. An antibody conjugated to a nonspecific fluorochrome of CD81 or LDL-R was used as negative control. The cells were then analyzed by FACS. The experiments were carried out at least 3 times.

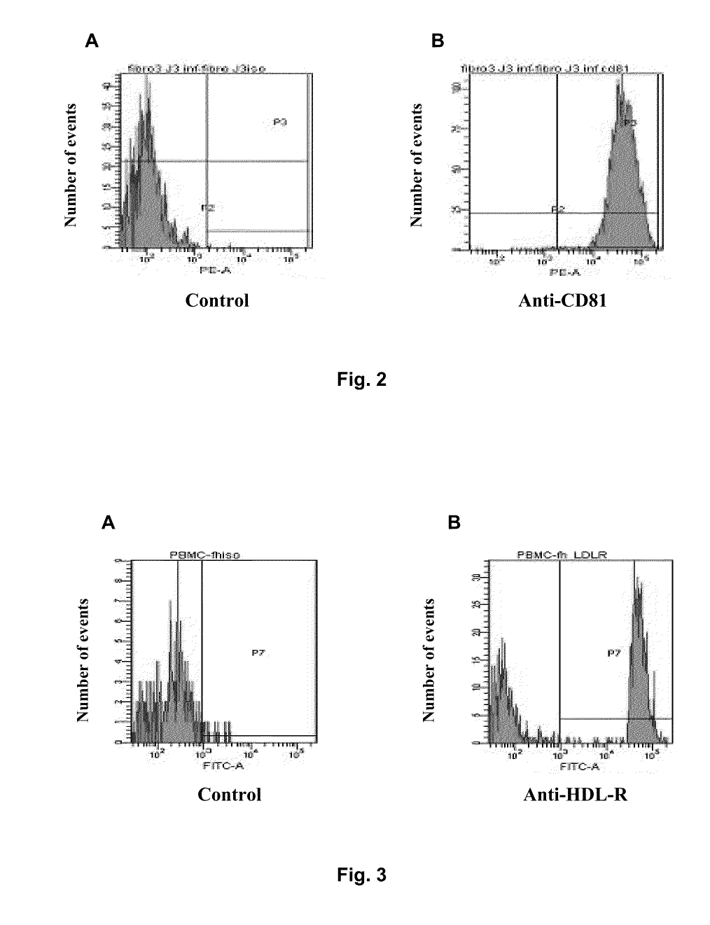

The results of analysis by flow cytometry obtained for CD81 and LDL-R are shown in FIGS. 2 and 3 respectively. In these figures, the number of events (number of cells) is shown on the ordinate, and the intensity of fluorescence (corresponding to labeling of the cells with the anti-CD81 antibody or anti-LDL-R antibody) is shown on the abscissa.

Analysis of the distribution peak indicates that more than 95% of the intrahepatic fibroblasts are labeled with the anti-CD81 antibody (cf. FIG. 2.A), and more than 70% are labeled with the anti-LDL-R antibody (cf. FIG. 3.A), whereas the negative controls do not show any labeling (FIGS. 2.B and 3.B).

These results confirm that the intrahepatic fibroblasts express CD81 and LDL-R on their cell surface.

### EXAMPLE 3

### Evaluation of Infection of Human Intrahepatic Fibroblasts (IHFs) with HCV

3.1. Preparation of Stocks of HCV-JFH1

Huh-7 or Huh-7.5.1 hepatocellular lines (thong et al. P.N.A.S., 102: 9294-9299) were grown at 37° C. in a humid atmosphere and 5% CO₂ in DMEM medium (Invitrogen) supplemented with 10 mM of HEPES (pH 7.3), nonessential amino acids (Invitrogen), 2 mM of L-glutamine (Invitrogen), and 10% of inactivated fetal calf serum. The HCV viral particles obtained from infection of Huh-7 cells were prepared using the method described by Wakita et al. (Nat. Med., 11: 791-796, 2005). Briefly, the cells were transfected with the genomic RNA obtained by transcription in vitro from the plasmid pJFH1. The culture supernatant is taken after several passages when the viral titer reaches at least 1.10⁵ focus-forming units per ml. Titration is performed as described by Pene et al. (J. Virol. Hepat., 2009). For certain preparations of HCV-JFH1, additional amplification of the virus was carried out by infecting Huh-7.5.1 cells. The stocks of supernatant containing the viruses were filtered on 0.45 μm membranes and then concentrated by ultrafiltration on a membrane having a cutoff of 100 000 dalton. The viral stocks were then divided into aliquots and stored at −80° C. The stocks were titrated as described by Pene et al. (J. Virol. Hepat., 2009). Briefly, Huh-7.5.1 cells were grown in the presence of a serial dilution of the filtered viral stocks and then, 3 days post-inoculation, infection of the cells was evaluated by immunofluorescence in situ. The cells infected with HCV appear as small cellular clusters, each cluster being regarded as an infectious focus. The infectious titer is expressed in focus-forming units (abbreviated hereinafter to “FFU”) per ml of stocks, determined by the number of foci at the highest dilution.

3.2. Infection of Intrahepatic Fibroblasts with HCV

The intrahepatic fibroblasts were infected one day after being put in culture with HCV-JFH1 to a multiplicity of infection of 0.1 FFU per cell. After incubation overnight at 37° C., the cellular monolayers were washed three times with phosphate-buffered saline and then grown in standard conditions (Aoudjehane et al., Lab. Invest., 88: 973-985, 2008) in DMEM medium (Gibco) 10% FCS in the presence of penicillin and streptomycin (1%) as well as pyruvate (1%).

A. Analysis of Replication of the HCV Genome in Intrahepatic Fibroblasts

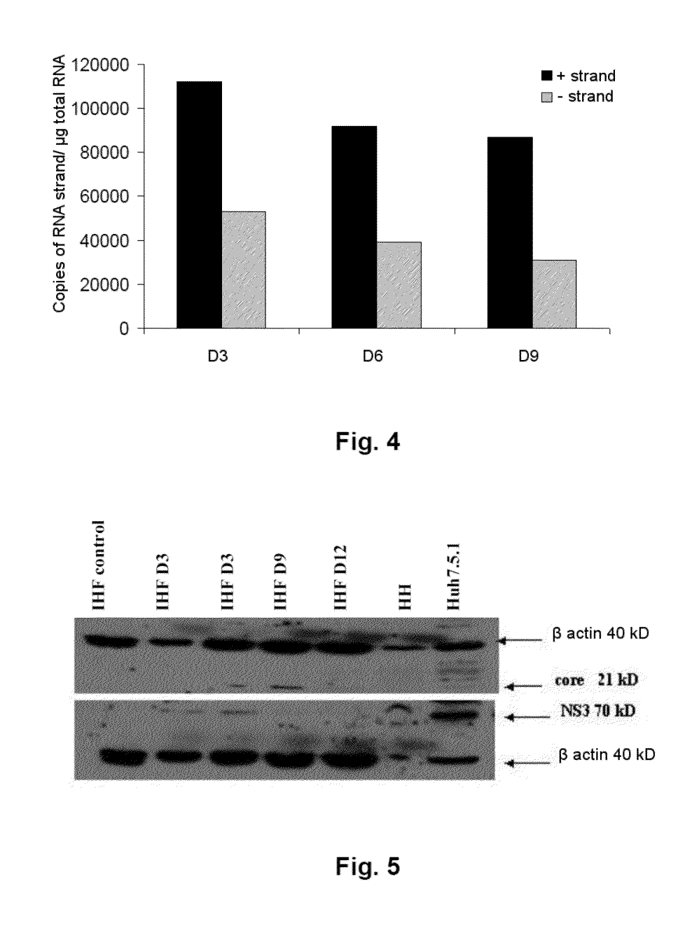

In order to determine whether the HCV genome is capable of replicating in intrahepatic fibroblasts inoculated with HCV-JFH1, the strands of positive RNA (genomic RNA) and the strands of negative RNA (antisense RNA) were quantified. For this purpose, the cells were taken on D3, D6 and D9 post-infection, washed in PBS buffer, then prepared using the “RNeasy minikit” extraction kit (QIAGEN S.A, Courtaboeuf, France) according to the supplier's recommendations, the cells having been lysed in 300 μl of buffer for 3.10⁵ cells. The intracellular RNAs were quantified by quantitative RT-PCR specific to each strand as described by Carriere et al. (J. Med. Virol., 79: 155-160, 2007). The results of this quantification are presented in FIG. 4.

FIG. 4 is a histogram showing the results obtained for quantification of the positive RNA strand (black bars) and quantification of the negative RNA strand (gray bars). The copy number of the RNA strand per μg of total RNA is shown on the ordinate, and the number of days post-inoculation is shown on the abscissa.

These results show that the positive RNAs, but also the negative RNAs (RNAs present only when replication of HCV takes place), are detectable starting from 3 days post-inoculation. This clearly shows that the HCV is capable of replicating effectively in intrahepatic fibroblasts.

B. Analysis of Expression of the HCV Genome in Intrahepatic Fibroblasts

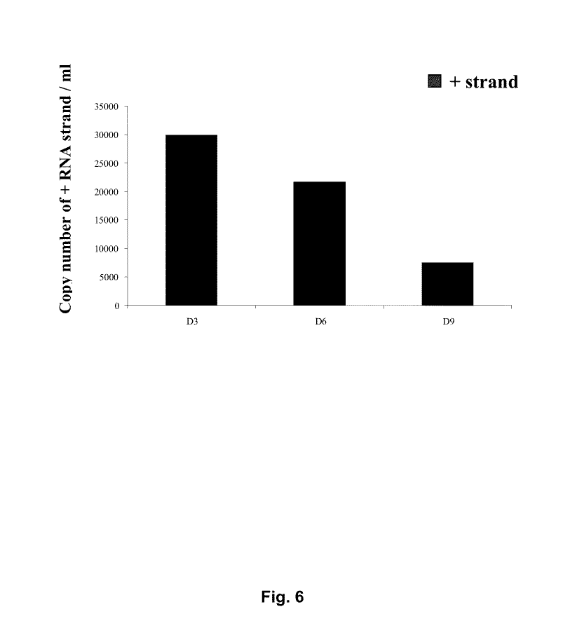

To determine whether the HCV polyprotein is translated and matured in intrahepatic fibroblasts, the expression of protease NS3 and of the HCV capsid protein was analyzed by Western blotting. Infected or uninfected (control) intrahepatic fibroblasts were washed in cold PBS and then lysed in the culture dishes by adding Laemmli buffer comprising 1.2% of β-2 mercaptoethanol (40 mM of Tris-HCl, pH 6.8, 5 mM of DTT, 1% of SDS, 7.5% of glycerol and 0.01% of bromophenol blue). After boiling the cellular lysate, the samples (comprising 50 mg of total proteins per well) were left to migrate in reducing conditions on 15% polyacrylamide-sodium dodecyl sulfate gel (for analysis of the capsid protein) or on 10% polyacrylamide gel (for analysis of protein NS3), then transferred to nitrocellulose membranes. The nitrocellulose membranes were saturated in PBS comprising 5% of milk, 0.1% of Tween 20 for 1 hour, then incubated for 1 hour with the mouse anti-HCV capsid monoclonal antibody (C7-50 antibody, Alexis Biochemicals) diluted to 1:1000 or with a mouse anti-HCV NS3 antibody (antibody 1847, Virostat, Portland, Mass.) diluted to 1:100. The membranes were then washed, incubated for 1 hour with an anti-mouse secondary antibody coupled to peroxidase diluted to 1:5000, washed and then incubated with a chemiluminescent reagent (Pierce, Rockford, Ill., USA). The results are shown in FIG. 5.

FIG. 5 shows that the capsid protein (“core”) is detectable on D3 and D9. This protein, which appears in the form of a single band of 21 kDa, is probably the mature form of the capsid. The protease NS3 is detectable on D3 and has the expected molecular weight (70 kDa).

This experiment confirms that the HCV polyprotein is expressed and correctly matured in intrahepatic fibroblasts inoculated with HCV-JFH1.

C. Analysis of the Infectious Potential of the Culture Supernatants

As the preceding results demonstrate that the HCV genome is capable of replicating and of expressing the HCV proteins in intrahepatic fibroblasts, the capacity of HCV to multiply and produce new infectious particles was investigated. For this purpose, the presence of positive and negative RNA in the culture supernatant of infected cells as described above was determined. FIG. 6 is a histogram showing the results obtained for quantification of the positive RNA strand. The copy number of the positive RNA strand per ml of supernatant is shown on the ordinate, and the number of days post-inoculation is shown on the abscissa. At three days post-infection, the positive RNA (genomic RNA) was detected, the amount decreasing with time. In contrast, the negative RNA strand, which is never present in the viral particles, was not detected. This indicates that the genomic RNA present in the supernatant was not salted-out in the medium by a nonspecific mechanism, without which the negative RNA would probably also have been detected.

However, the supernatant proved to be noninfectious on inoculation of Huh-7.5.1 cells.

### EXAMPLE 4

### Neutralization of the Infection of Human Intrahepatic Fibroblasts (IHFs)

4.1. Test of Neutralization by Anti-CD81 Antibodies

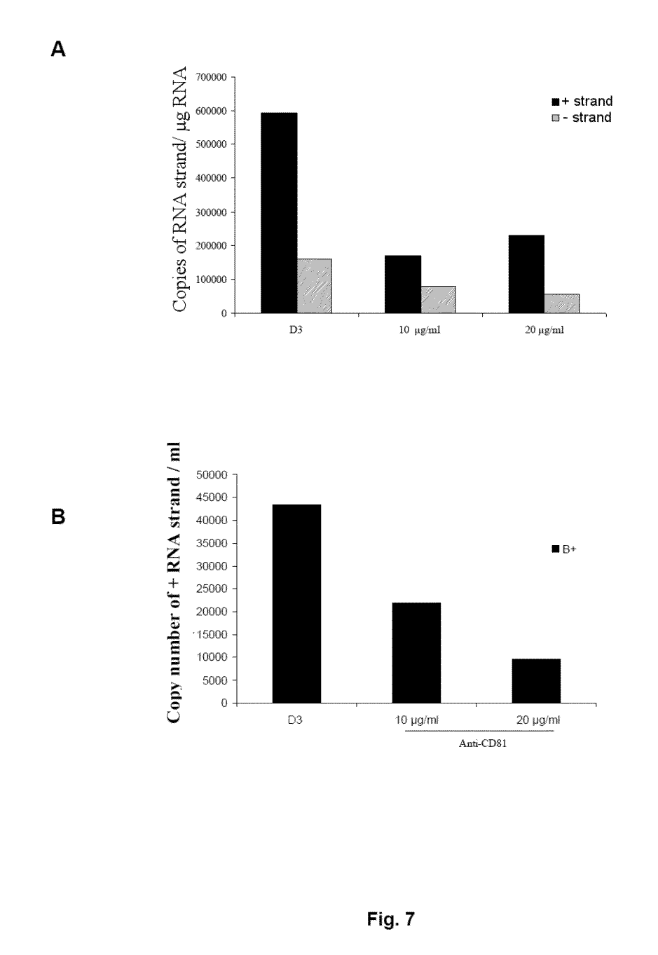

Numerous results published in recent years show that the CD81 molecule should be a surface receptor of HCV essential to its attachment to and entry into the cell. In order to determine whether the infection of intrahepatic fibroblasts with HCV-JFH1 depends on the CD81 surface molecule, intrahepatic fibroblasts were preincubated for one hour at 37° C. with increasing amounts of monoclonal mouse anti-human CD81 antibody or of nonspecific antibody (control). The cells were washed and then infected with HCV-JFH1 at a multiplicity of infection of 0.1 FFU per cell. To evaluate the percentage neutralization, the amounts of positive and negative intracellular RNAs, as well as the amount of extracellular positive RNA in the supernatant, were determined at three days post-infection as described in section 3.2.A. The results are presented in FIG. 7.

FIG. 7.A is a histogram showing the results obtained for quantification of the positive intracellular RNA strand (black bars) and quantification of the negative intracellular RNA strand (gray bars). The copy number of the RNA strand per μg of total RNA is shown on the ordinate, and the concentration of anti-CD81 is shown on the abscissa. This diagram shows a large reduction in the amount of positive-strand RNA and of negative-strand RNA when the cells are preincubated with 10 μg of anti-CD81 per ml of culture medium relative to the control: infected intrahepatic fibroblasts incubated with a nonspecific antibody taken at 3 days post-inoculation (bars D3). Moreover, FIG. 7.B, which is a histogram showing the results obtained for quantification of the positive extracellular RNA strand, shows that there is a dose-dependent decrease in the amount of positive RNA in the culture supernatant.

These results show that infection of intrahepatic fibroblasts with HCV-JFH1 is predominantly dependent on the presence of CD81 on the surface of the cells, which indicates that HCV infects the cells using CD81 as the main cellular receptor.

4.2. Test of Neutralization by Interferon Alfa

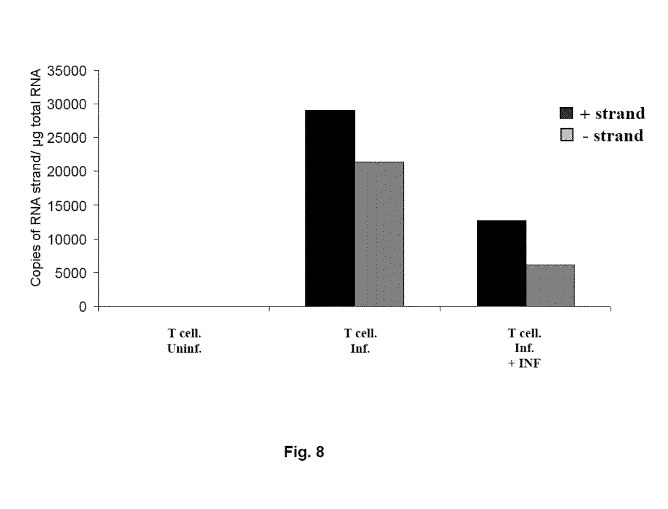

Since interferon alfa inhibits the replication of HCV in infected hepatocytes in vitro and has been used for many years for treating patients who have hepatitis C, intrahepatic fibroblasts were preincubated for one hour at 37° C. in the presence of 500 U/ml of interferon alfa, washed and then infected with HCV-JFH1 to a multiplicity of infection of 0.1 FFU per cell. The results are presented in FIG. 8. This diagram shows that the amounts of positive-strand RNA and of negative-strand RNA decrease when the cells are preincubated with interferon alfa.

### EXAMPLE 5

### Evaluation of Apoptosis Induced During Infection of Human Intrahepatic Fibroblasts (IHFs) with HCV

In order to determine whether HCV infection induces apoptosis of intrahepatic fibroblasts, an Annexin V-FITC in vitro apoptosis assay was performed using the Annexin V-FITC kit (Immunotech, Marseilles, France) according to the supplier's instructions. This assay is based on externalization of phosphatidylserine by the apoptotic cells, and on fixation of Annexin V-FITC on this molecule. Necrosis was also determined by counterstaining with propidium iodide. After culture for 48 hours, the intrahepatic fibroblasts were either infected with HCV-JFH1 to a multiplicity of infection of 0.1 FFU per cell, or treated with 20 μM of C2-Ceramide (Sigma) (positive control of apoptosis). After 3, 6 and 9 days post-infection, the cells were suspended in a binding buffer containing Ca²⁺ and incubated with 1 μg/ml of Annexin C-FITC and 1 μg/ml of propidium iodide. The signal emitted by the FITC (fluorescein isothiocyanate) was then measured by flow cytometry to determine apoptosis, whereas necrosis was determined by measuring the number of cells labeled with propidium iodide. It was found that infection with HCV-JFH1 induces apoptosis of a minimal number of intrahepatic fibroblasts and that the infection is not toxic for the intrahepatic fibroblasts (results not shown).

### EXAMPLE 6

### Effect of Infection with HCV on the Proliferation of Human Intrahepatic Fibroblasts and Expression of the Genes Involved in Fibrosis

6.1. Evaluation of Proliferation of Intrahepatic Fibroblasts Following HCV Infection

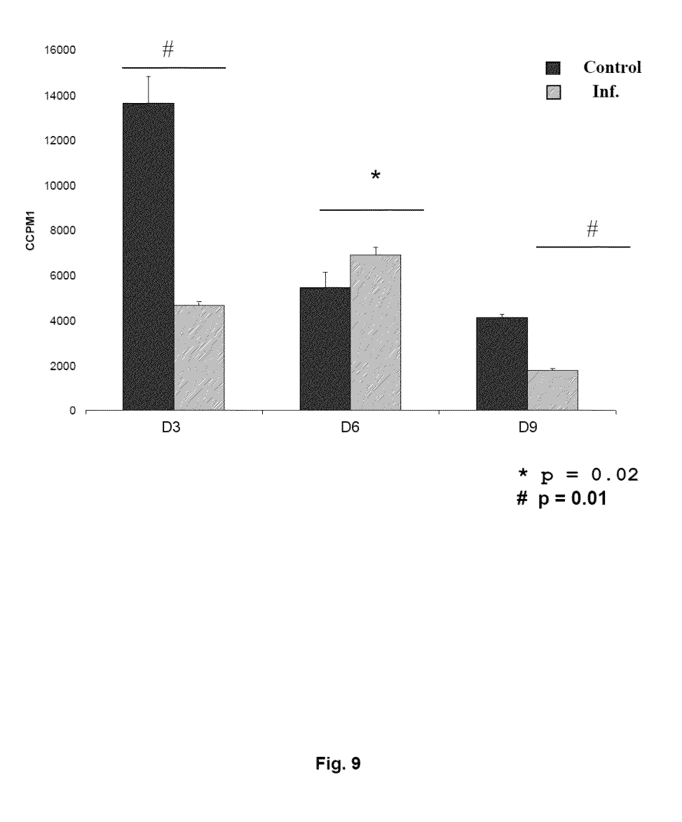

The intrahepatic fibroblasts were seeded in 96-well plates (5.10⁴ cells per well) in serum-free DMEM medium (Gibco). At 48 post-seeding, the cells were infected with HCV-JFH1 to a multiplicity of infection of 0.1 FFU per cell. A control with uninfected cells was also carried out. Cellular proliferation was measured after 16 hours of culture in the presence of tritiated [3H] thymidine (1 μCi/well) by means of a scintillation counter. These results are presented in FIG. 9.

FIG. 9 is a histogram in which the control with uninfected cells is represented by the black bars and the tests relating to the infected cells are represented by the gray bars. The number of pulses per minute (“CCPM1”) is shown on the ordinate, and the number of days post-infection is shown on the abscissa. This diagram shows that proliferation is significantly inhibited (P=0.01) at three days post-infection relative to the control with uninfected cells. However, at 6 days post-infection the infected cells showed significantly more proliferation than the control (p=0.02). At 9 days post-infection, the cells are confluent and the results can no longer be interpreted.

6.2. Analysis of Expression of the Genes of Collagen Type I and Type IV, and of Smooth Muscle Alpha-Actin in HCV-Infected Intrahepatic Fibroblasts

Expression of the genes of GAPDH, of collagen type I, of collagen type IV, of smooth muscle alpha-actin, as well as expression of the genes of HNF-1β, of cytochrome P450 and of albumin were analyzed by RT-PCR using the “DNA Fast Start SYBR green” kit (Roche Diagnostics), and the LightCycler® equipment (Roche Diagnostics), in intrahepatic fibroblasts, uninfected (control) or infected with HCV.

PCR amplification was performed in a total volume of 20 μl/ml in capillary containing 20 ng of each primer of the primer pairs described in Table I below (Sigma-Genosys Ltd), 3 mM of MgCl₂, 2 μl of Light Cycler Fast Start Master SYBR Green composition (containing 1.25 units of Fast StartTaq polymerase, Taq 10× buffer, 2 mM of each of the dNTPs, 10 μl of SYBR Breen 10×(Roche Diagnostics) and 2 μl of cDNA (previously diluted to 1/10) derived from each strain and obtained as described by Zhong et al. (P.N.A.S., 102: 9294-9, 2005) et al. (J. Med. Virol., 79: 155-60, 2007).

TABLE I

| | Primers: sense sequence (+) | SEQ |
|---|---|---|
| Gene | Antisense sequence (-) | ID(#) |
| GAPDH | (+)5′-ACAGTCCATGCCATCACTGCC-3′ | (1) |
| | (-)5′-GCCTGCTTCACCACCTTCTTG-3′ | (2) |
| collagen type I | (+)5′-CCTCAAGGGCTCCAACGAG-3′ | (3) |
| | (-)5′-TCAATCACTGTCTTGCCCCA-3′ | (4) |
| collagen type IV | (+)5′-ATGTCAATGGCACCCATCAC-3′ | (5) |
| | (-)5′-CTTCAAGGTGGACGGCGTAG-3′ | (6) |
| smooth muscle | (+)5′-TGAAGAGCATCCCACCCT-3′ | (7) |
| alpha-actin | (-)5′-ACGAAGGAATAGCCACGC-3′ | (8) |
| HNF-1β | (+)5′-GAAACAATGAGATCACTTCCTC-3′ | (9) |
| | (-)5′-CTTTGTGCAATTGCCATGACTC-3′ | (10) |
| Cytochrome P450 | (+)5′-AGCACAACTCTGAGATATGG-3′ | (11) |
| | (-)5′-ATAGTCACTGTACTTGAACT-3′ | (12) |
| Albumin | (+)5′-TTAGGATCCCCCAGGAAGACATCCTTTGC-3′ | (13) |
| | (-)5′-CCTGAGCCAGAGATTTCC-3′ | (14) |

Amplification was carried out in the following conditions:
initial denaturation at 94° C. for 10 min,
40 cycles of amplification each comprising 3 phases:
   i) 95° C. for 10 s (denaturation);
   ii) hybridization of the primers with DNA for 5 s at 70° C. for GAPDH, 58° C. for collagen type I, 65° C. for collagen type IV, 63° C. for smooth muscle alpha-actin, 66° C. for HNF-1β, 60° C. for CP450, 70° C. for albumin;
   iii) 72° C. for 5 s (polymerization).

The relative level of expression of the mRNAs of collagen IV, of smooth muscle alphβ-actin, of HNF-1β, of CP450 and of albumin was calculated using the “2^{ΔΔCT” method described by Livak and Schmittgen (Methods,} 25: 402-408, 2001).

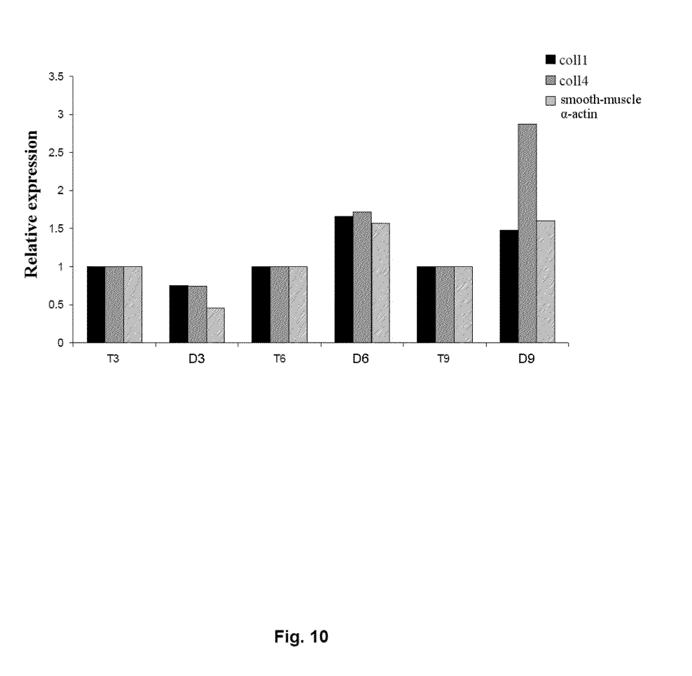

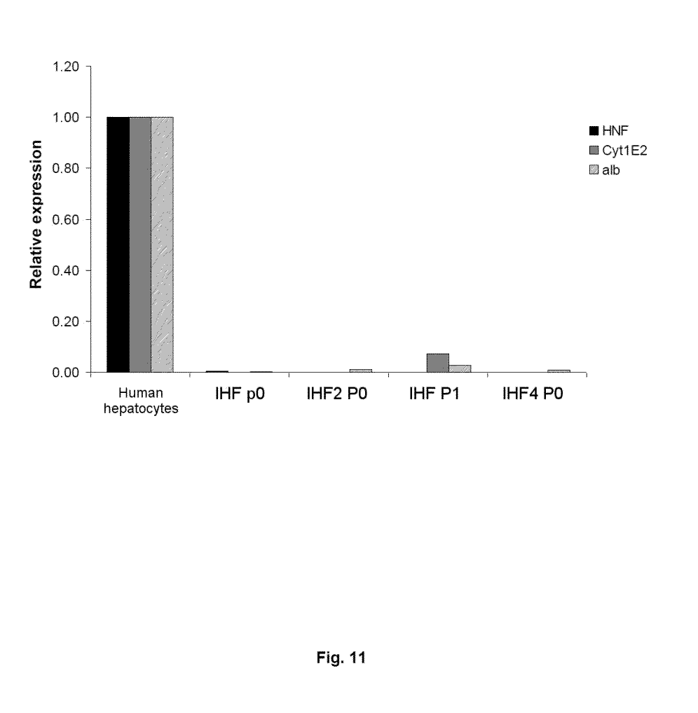

The results of this experiment are presented in FIG. 10. The expression of collagen type I, of collagen type IV and of smooth muscle alpha-actin is shown by the black, dark gray and light gray bars respectively. The relative expression is shown on the ordinate. The origin of the sample, namely uninfected cells (control) or infected cells, is shown on the abscissa (“T” for control and “D” for tests with infected cells). The day post-infection when the sample was taken is also shown. This diagram shows that on D3, expression of collagen type I, of collagen type IV and of smooth muscle alpha-actin by the infected cells is less than that of the uninfected cells. However, at 6 and 9 days post-infection, expression of these three genes is significantly greater than that of the control with uninfected cells (p=0.02).

The set of results presented in example 6 indicates that for intrahepatic fibroblasts infected with HCV-JFH1, cellular proliferation and activation are inhibited initially, but are then increased.

### EXAMPLE 7

### Isolation of Human Intrahepatic Fibroblasts Infected In Vivo with HCV

Intrahepatic fibroblasts were isolated from the liver of patients infected with HCV as described in example I. The purity of the intrahepatic fibroblasts was evaluated by measuring the expression of smooth muscle alpha-actin, a marker of hepatic stellate cells, and of vimentin, a marker of cells of mesenchymal origin. Moreover, the intrahepatic fibroblasts were also characterized by measuring the expression of CD90 and of CD31. In order to rule out contamination of the intrahepatic fibroblasts with infected hepatocytes, the mRNAs of three genes specifically expressed by the hepatocytes were quantified. These three genes are:

i) the gene of the HNF-4 (“hepatocyte nuclear factor”) transcription factor, a key regulator in maintaining the hepatocyte phenotype; ii) the gene of albumin, which is expressed in the primary hepatocytes;

iii) the CYP2E1 gene, a marker of the detoxification function of the hepatocytes.

The results are presented in FIG. 11. The number of passages is shown on the abscissa (“p0”, “p1”). IHFs, IHF2 and IHF4 represent different batches of intrahepatic fibroblasts. As this diagram shows, whereas the human hepatocyte (“HH”) control expresses each of these genes, the intrahepatic fibroblasts do not express any of these 3 genes, even after one passage.

The intrahepatic fibroblasts isolated from the liver of patients infected with HCV therefore were not contaminated with infected hepatocytes during isolation.

In order to determine whether the intrahepatic fibroblasts isolated from HCV-positive patients are infected with HCV, the positive and negative intracellular

RNAs of HCV were quantified as described in example 3.2.A. Moreover, the positive RNA was quantified in the supernatant.

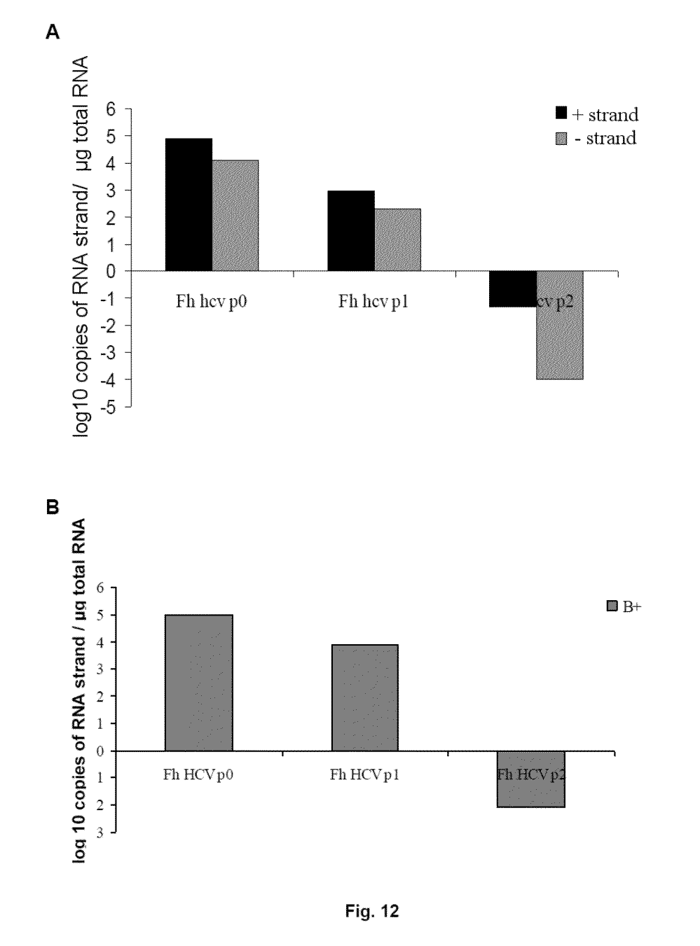

The results are presented in FIG. 12. In FIG. 12.A, which gives the results of quantification of intracellular viral RNAs, the copy number of positive RNA (black bars) and of negative RNA (gray bars) is shown on the ordinate (in log 10 per μg of total RNA), and the number of passages is shown on the abscissa (“p0”, “p1”, “p2”). This diagram shows that the intrahepatic fibroblasts isolated express about 1.10⁵ strands of positive RNA per μg of total RNA and are therefore well infected in vivo with HCV. Moreover, the detection of 1.10⁴ strands of negative RNA per μg of total RNA clearly shows that HCV replicates in the intrahepatic fibroblasts in vivo. After one passage of the cells, the positive RNA number decreases, and continues to decrease during the successive passages.

FIG. 12.B shows the results obtained for quantification of the positive RNA strand in the supernatant. The copy number of the positive RNA strand per ml of supernatant is shown on the ordinate, and the passage number is shown on the abscissa. The genomic RNA is certainly present in the supernatant, as the evolution of the number of positive strands follows that observed in the case of intracellular viral RNAs.

1. A method of obtaining intrahepatic fibroblasts infected with the hepatitis C virus, comprising a step of contacting intrahepatic fibroblasts in vitro with infectious particles of the hepatitis C virus and isolating infected intrahepatic fibroblasts.

2. The method as claimed in claim 1, characterized in that the hepatitis C virus is HCV-JFH1.

3. A method of obtaining intrahepatic fibroblasts infected with the hepatitis C virus, comprising a step of isolating intrahepatic fibroblasts from a sample of a hepatic tissue previously taken from an HCV-positive patient and isolating infected intrahepatic fibroblasts.

4. An isolated intrahepatic fibroblast infected with a hepatitis C virus, or comprising the genome of a hepatitis C virus or a replicon thereof, obtainable the method defined in claim 1.

5. A method of replication in vitro of the genome of the hepatitis C virus, characterized in that it comprises a step of culture in vitro of isolated intrahepatic fibroblasts infected with the hepatitis C virus.

6. The method as claimed in claim 5, characterized in that the isolated intrahepatic fibroblasts infected with the hepatitis C virus are obtained by the method defined in claim 1.

7. A method of screening anti-HCV molecules comprising the following steps:
contacting intrahepatic fibroblasts infected with a hepatitis C virus or intrahepatic fibroblasts comprising the genome of a hepatitis C virus or a replicon thereof with the molecule to be tested;
measuring the replication of the HCV genome and/or the production of HCV particles.

8. The method of screening as claimed in claim 7, characterized in that the step of measuring the replication of the HCV genome is performed by quantification of the RNA strand of positive polarity and/or the RNA strand of negative polarity of the HCV.

9. A method of screening antifibrogenesis molecules comprising the following steps:
contacting intrahepatic fibroblasts infected with a hepatitis C virus or intrahepatic fibroblasts comprising the genome of a hepatitis C virus or a replicon thereof with the molecule to be tested;
measuring the expression of at least one gene implicated in fibrosis.

10. The method of screening as claimed in claim 9, characterized in that the gene or genes implicated in fibrosis whose expression is measured are selected from the genes of collagens type I, III, IV and V.

11. A method of evaluating the toxicity of a molecule with respect to hepatic fibroblasts of a patient with hepatitis C comprising the following steps:
contacting intrahepatic fibroblasts infected with a hepatitis C virus or intrahepatic fibroblasts comprising the genome of a hepatitis C virus or a replicon thereof with the molecule to be tested;
evaluating the mortality of said intrahepatic fibroblasts.


SEQUENCE LISTING (14 sequences)

SEQ ID NO: 1 - DNA, 21 bp, Artificial
  GAPDH sense primer
acagtccatgccatcactgcc

SEQ ID NO: 2 - DNA, 21 bp, Artificial
  GAPDH antisense primer
gcctgcttcaccaccttcttg

SEQ ID NO: 3 - DNA, 19 bp, Artificial
  Collagen Type I sense primer
cctcaagggctccaacgag

SEQ ID NO: 4 - DNA, 20 bp, Artificial
  Collagen Type I antisense primer
tcaatcactgtcttgcccca

SEQ ID NO: 5 - DNA, 20 bp, Artificial
  Collagen Type IV sense primer
atgtcaatggcacccatcac

SEQ ID NO: 6 - DNA, 20 bp, Artificial
  Collagen Type IV antisense primer
cttcaaggtggacggcgtag

SEQ ID NO: 7 - DNA, 18 bp, Artificial
  Smooth-muscle alpha-actin sense primer
tgaagagcatcccaccct

SEQ ID NO: 8 - DNA, 18 bp, Artificial
  Smooth-muscle alpha-actin antisense primer
acgaaggaatagccacgc

SEQ ID NO: 9 - DNA, 22 bp, Artificial
  HNF-1 beta sense primer
gaaacaatgagatcacttcctc

SEQ ID NO: 10 - DNA, 22 bp, Artificial
  HNF-1 beta antisense primer
ctttgtgcaattgccatgactc

SEQ ID NO: 11 - DNA, 20 bp, Artificial
  CP450 sense primer
agcacaactctgagatatgg

SEQ ID NO: 12 - DNA, 20 bp, Artificial
  CP450 antisense primer
atagtcactgtacttgaact

SEQ ID NO: 13 - DNA, 29 bp, Artificial
  Albumin sense primer
ttaggatcccccaggaagacatcctttgc

SEQ ID NO: 14 - DNA, 18 bp, Artificial
  Albumin antisense primer
cctgagccagagatttcc